(19) United States
(12) Patent Application Publication
(10) Pub. No.: US 2023/0181103 A1
(43) Pub. Date: Jun. 15, 2023

(54) APPARATUS AND METHOD FOR EVALUATING OBSTRUCTIVE SLEEP APNEA WITH PPG SIGNAL

(71) Applicant: ACME PORTABLE CORP., New Taipei (TW)

(72) Inventors: Yii-leh Kao, New Taipei (TW); Tsair Kao, New Taipei (TW); Yin-tsong Lin, New Taipei (TW); Tun-hou Lin, New Taipei (TW); Ching-chih Lin, New Taipei (TW)

(73) Assignee: ACME PORTABLE CORP., New Taipei (TW)

(21) Appl. No.: 17/822,785

(22) Filed: Aug. 28, 2022

(30) Foreign Application Priority Data
Dec. 10, 2021 (TW) 110146466

(51) Int. Cl. A61B 5/00; A61B 5/0205; A61B 5/11

(52) U.S. Cl. CPC A61B 5/4818; A61B 5/0205; A61B 5/1114; A61B 5/746; A61B 5/6826; A61B 5/4809; A61B 5/02416

(57) ABSTRACT

An apparatus and method for evaluating obstructive sleep apnea with PPG signal are provided. Measured heartbeat interval signal is used to evaluate whether there is obstructive apnea. The system includes a motion sensor for detecting whether a user is in a stationary state; an optical sensor for measuring the heartbeat interval signal of the user in a stationary state; a microprocessor for processing the heartbeat interval signal of the user to obtain an apnea parameter; an alert module for receiving the apnea parameter and feeding them back to the user; and a memory module for storing the apnea parameter after the signal processing. The system and method are used to determine whether the heartbeat interval signal is an obstructive sleep apnea signal, and further determine whether the user is in an obstructive sleep apnea situation.

### CROSS REFERENCE TO RELATED APPLICATION

This application claims priority to Taiwan Patent Application No. 110146466, filed on Dec. 10, 2021, which is incorporated herein by reference in its entirety.

### FIELD OF INVENTION

This disclosure relates to a physiological signal analysis device, and more particularly, to an apparatus and method for evaluating obstructive apnea with PPG signal.

### BACKGROUND OF INVENTION

Sleep apnea is a kind of sleep breathing disorder. Obstructive Sleep Apnea (OSA) is a common sleep-disordered breathing characterized by a decrease or cessation of respiratory airflow for a period of time due to complete or partial obstruction of the upper respiratory tract during sleep.

In addition, for cardiovascular-related diseases, atrial fibrillation (AF) is the most common arrhythmia in clinical practice. At present, it is clinically observed that atrial fibrillation (AF) is highly associated with obstructive apnea (OSA), and the recurrence rate of patients with atrial fibrillation and obstructive apnea after the rehabilitation of atrial fibrillation is much higher than that of patients with obstructive apnea only.

At present, there are two main methods for heart rate detection, which include “light transmission measurement method” and “electrical signal measurement method”. The light transmission measurement method is also known as photoplethysmography (PPG). The light transmission measurement method is to project a light beam on the skin and measure the reflected or transmitted light signal to obtain the condition of the heartbeat. The principle of the electrical signal measurement method is similar to ECG. The method uses a sensor to directly measure the electrical signal generated when the myocardium contracts to determine the user's heart rate. In the current technology, no matter whether the “light transmission measurement method” or the “electric signal measurement method” is used, how to quickly and accurately analyze and interpret the detected signal after the signal detection is the key to the accuracy of the physiological signal analysis device. Therefore, while determining the heart rate of the user, how to more accurately detect whether the user suffers from obstructive apnea from the heart rate detection signal is the technical problem to be solved by the present application.

### SUMMARY OF INVENTION

Conventional devices/methods lack the technology capable of detecting both atrial fibrillation (AF) and obstructive apnea (OSA). The disclosure provides an apparatus and method that can detect and evaluate atrial fibrillation and obstructive apnea on the same device. For patients suffering from both atrial fibrillation and obstructive apnea, the apparatus and the method for detecting and evaluating atrial fibrillation and obstructive apnea simultaneously can be provided after the atrial fibrillation has been cured.

In order to solve the above drawbacks, the disclosure provides an apparatus for evaluating obstructive apnea with PPG signal. The apparatus comprises:

a motion sensor configured to detect whether a user is in a stationary state;

an optical sensor configured to measure a heartbeat interval signal of the user in the stationary state, wherein the heartbeat interval signal is the PPG signal;

a microprocessor, electrically connected to the motion sensor and the optical sensor, respectively, and configured to perform a signal processing on the heartbeat interval signal of the user to obtain an apnea parameter;

an alert module, electrically connected to the microprocessor, configured to receive the apnea parameter and configured to issue an alert; and

a memory module electrically connected to the microprocessor and configured to store the apnea parameter after the signal processing.

The signal processing comprises: a first analysis program for extracting a pulse rate interval sequence and a respiration sequence from the heartbeat interval signal, respectively; a second analysis program for calculating a cross-power spectral density of the pulse rate interval sequence and the respiration sequence to obtain a first parameter, calculating a coherence between the pulse rate interval sequence and the respiration sequence to obtain a second parameter, and calculating a spectral power of the respiration sequence to obtain a third parameter, wherein the first parameter and the second parameter are multiplied, and a multiplied value of the first parameter and the second parameter in a low frequency band and a high frequency band, respectively, are extracted to obtain a high frequency pulse/respiration signal (HFpulse/resp) and a low frequency pulse/respiration signal (LFpulse/resp), and values of the third parameter in the low frequency band and the high frequency band, respectively, are extracted to obtain a high frequency respiration signal (HFresp) and a low frequency respiration signal (LFresp); and a third analysis program for multiplying a ratio of the low frequency pulse/respiration signal to the high frequency pulse/respiration signal (LFpulse/resp/HFpulse/resp) and a ratio of the low frequency respiration signal to the high frequency respiration signal (LFresp/HFresp) and performing a logarithmic processing to obtain the apnea parameter, and for comparing the apnea parameter with a threshold in a normal sleep respiration database to determine whether the heartbeat interval signal is an obstructive apnea signal, and further determine whether the user is in an obstructive apnea condition.

In order to solve the above drawbacks, the disclosure further provides a method for evaluating obstructive apnea with PPG signal, which is characterized in applying the device for evaluating obstructive apnea with PPG signal, and comprises the following steps:

a step S01 of providing an apparatus for evaluating obstructive sleep apnea with the PPG signal for determining whether a user is in a stationary state;

a step S02 that when the user is determined in the stationary state, a heartbeat interval signal of a user is measured and obtained, wherein the heartbeat interval signal is a PPG signal;

a step S03 of performing a signal processing on the heartbeat interval signal of the user to obtain an apnea parameter;

the signal processing comprises:

a first analysis program for extracting a pulse rate interval sequence and a respiration sequence from the heartbeat interval signal, respectively;

a second analysis program for calculating a cross-power spectral density of the pulse rate interval sequence and the respiration sequence to obtain a first parameter, calculating a coherence between the pulse rate interval sequence and the respiration sequence to obtain a second parameter, and calculating a spectral power of the respiration sequence to obtain a third parameter, wherein the first parameter and the second parameter are multiplied, and a multiplied value of the first parameter and the second parameter in a low frequency band and a high frequency band, respectively, are extracted to obtain a high frequency pulse/respiration signal (HFpulse/resp) and a low frequency pulse/respiration signal (LFpulse/resp), and values of the third parameter in the low frequency band and the high frequency band, respectively, are extracted to obtain a high frequency respiration signal (HFresp) and a low frequency respiration signal (LFresp); and

a third analysis program for multiplying a ratio of the low frequency pulse/respiration signal to the high frequency pulse/respiration signal (LFpulse/resp/HFpulse/resp) and a ratio of the low frequency respiration signal to the high frequency respiration signal (LFresp/HFresp) and performing a logarithmic processing to obtain the apnea parameter, and for comparing the apnea parameter with a threshold in a normal sleep respiration database to determine whether the heartbeat interval signal is an obstructive apnea signal, and further determine whether the user is in an obstructive apnea condition; and

a step S04 of determining whether the apnea parameter is higher than a threshold, wherein when the apnea parameter is higher than the threshold, it is determined that the user is in an obstructive apnea situation, and the apparatus issues an alert, and when the apnea parameter is lower than the threshold, it is determined that the user is not in the obstructive apnea condition, and the apparatus does not issue an alert.

The disclosure provides an apparatus and method for evaluating obstructive apnea with PPG signal. For users suffering from both atrial fibrillation and obstructive apnea, atrial fibrillation and obstructive apnea can be detected and evaluated simultaneously. For users suffering from both atrial fibrillation and obstructive apnea, after the atrial fibrillation has been cured, the probability of recurrence is much higher than that of patients with obstructive apnea only. Therefore, the apparatus and method for evaluating obstructive apnea with PPG signal provided by the disclosure can measure the heartbeat interval signal of the user in a stationary state (such as a sleep state at night) to determine whether the user is in the state of obstructive apnea. The heart rate signal of the user is measured when the user is not sleeping to determine whether there is arrhythmia. The apparatus and the method can solve that the conventional device lacks technology capable of detecting both atrial fibrillation and obstructive apnea.

### DESCRIPTION OF DRAWINGS

In order to more clearly illustrate the embodiments of the disclosure or the technical solutions in the prior art, the following briefly introduces the accompanying drawings used in the embodiments. Obviously, the drawings in the following description merely show some of the embodiments of the disclosure. As regards one of ordinary skill in the art, other drawings can be obtained in accordance with these accompanying drawings without making creative efforts.

FIGS. 1A to 1C are structural schematic diagrams of an apparatus for evaluating obstructive apnea with PPG signals according to an embodiment of the disclosure.

FIGS. 2A to 2B are schematic diagrams of the use state of an apparatus for evaluating obstructive apnea with PPG signals according to the embodiment of the disclosure.

FIG. 3 is a block diagram of a module of the apparatus for evaluating obstructive apnea with PPG signals according to an embodiment of the disclosure.

FIG. 4 is a schematic flowchart of a method for evaluating obstructive apnea with PPG signals according to the disclosure.

FIG. 5 is a schematic flowchart of a signal processing step of the method for evaluating obstructive apnea with PPG signals according to the disclosure.

FIGS. 6A to 6B are the apparatus for evaluating obstructive apnea with PPG signals to measure physiological signals of a user under a normal sleep respiration condition (FIG. 6A) and a obstructive apnea condition (FIG. 6B), respectively, parts (a) in FIG. 6A and FIG. 6B represent pulse rate interval sequence (solid line) and respiration sequence (broken line), parts (b) represent pulse rate interval sequence spectrum (solid line) and respiration sequence spectrum (broken line), and parts (c) represent the product of the cross-power spectral density of the pulse rate interval sequence and the respiration sequence and the coherence of the pulse rate interval sequence and the respiration sequence.

### DETAILED DESCRIPTION OF PREFERRED EMBODIMENTS

The disclosure mainly discloses an apparatus and method for evaluating whether a user suffers from obstructive apnea by using a heartbeat-related signal (i.e., PPG signal). The optical sensor of the apparatus measures the user's heartbeat signal when the user is in a stationary state to evaluate whether the user suffers from obstructive apnea. The basic principles and functions of the apparatus and method related to the disclosure can be understood by those with ordinary skill in the relevant technical field. Therefore, the following description will only describe in detail the technical features related to the apparatus and method of the disclosure. In addition, the drawings for the specification are not completely drawn according to the actual relative dimensions, and their function is only to describe the schematic diagrams related to the characteristics of this disclosure.

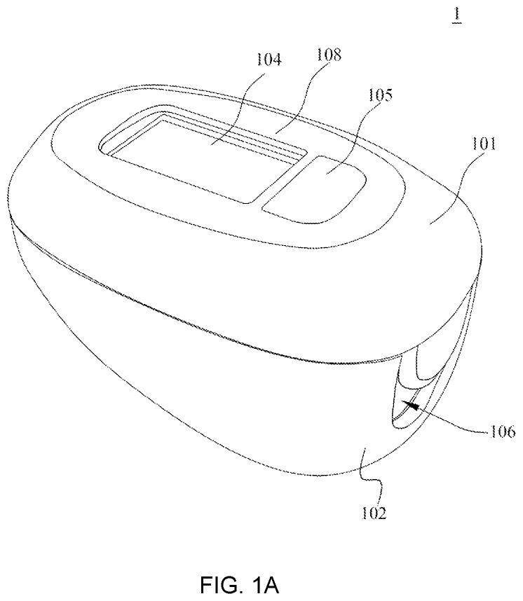

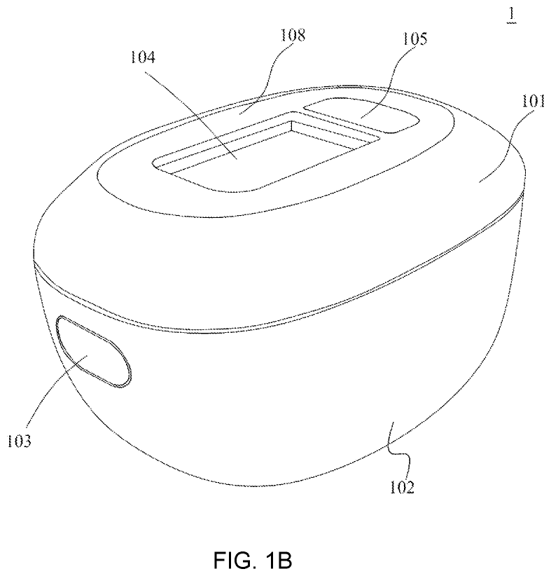

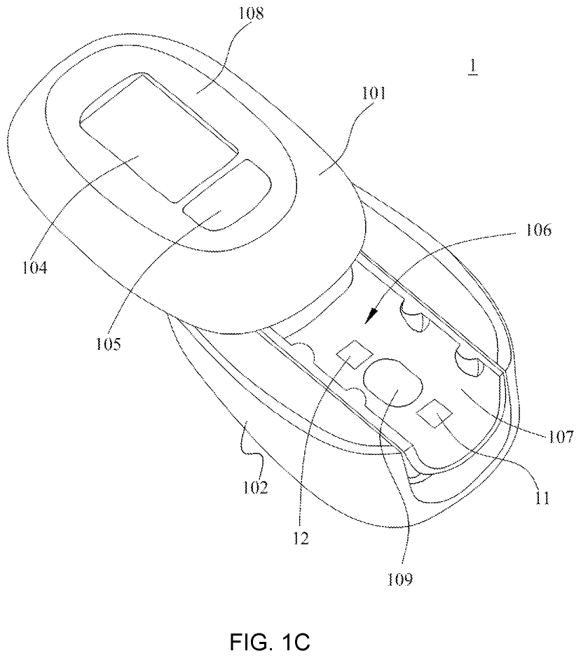

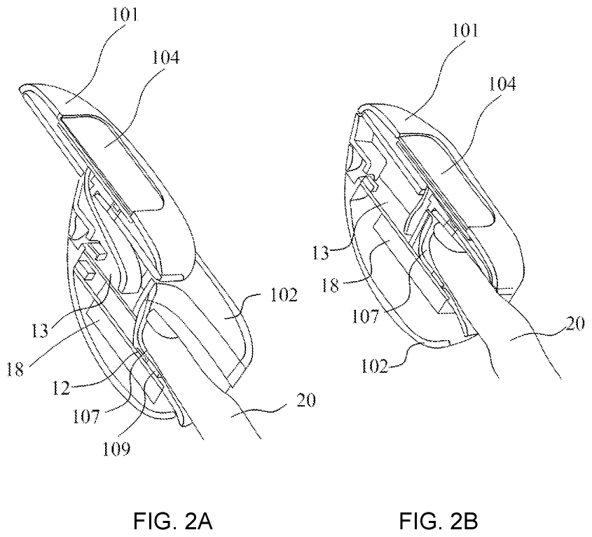

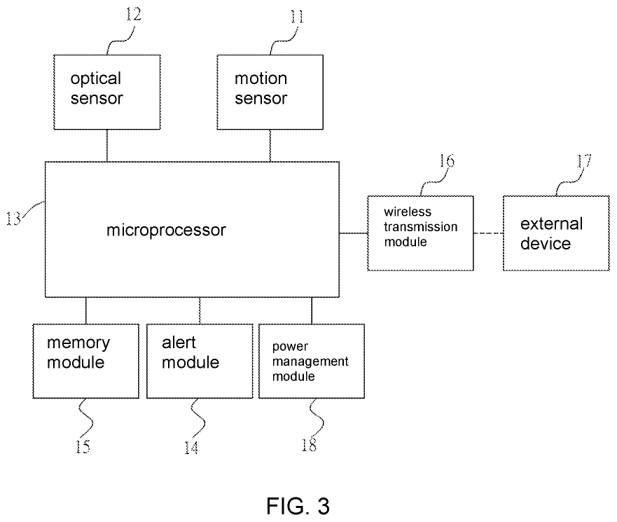

Referring to FIG. 1A to FIG. 1C, structural schematic diagrams of an apparatus for evaluating obstructive apnea with PPG signals according to an embodiment of the disclosure are shown in FIG. 1A to FIG. 1C, and referring to FIG. 2A to FIG. 2B and FIG. 3, schematic diagrams of the use state of an apparatus for evaluating obstructive apnea with PPG signals according to the embodiment of the disclosure and a block diagram of a module of the apparatus for evaluating obstructive apnea with PPG signals according to an embodiment of the disclosure are shown. The apparatus 1 of the disclosure includes a motion sensor 11, an optical sensor 12, a microprocessor 13, an alert module 14 and a memory module 15. The motion sensor 11, the optical sensor 12, the alert module 14, and the memory module 15 are respectively electrically connected to the microprocessor 13.

In an embodiment of the disclosure, the motion sensor 11 detects whether a user is in a stationary state when the user does not sleep, and transmits a signal representing the stationary state to the microprocessor 13. The stationary state is a state in which the user is in sleep. The motion sensor 11 can be an acceleration sensor, such as a linear accelerometer, or a gravity sensor (g sensor). In an embodiment of the disclosure, the optical sensor 12 refers to a sensing element that obtains an optical signal using the principle of PPG (photolithography). For example, the measurement is carried out by means of light penetration or light reflection to obtain the user's blood physiological information, so other physiological information can be further analyzed and obtained. For example, the information of the change of blood oxygen concentration can be obtained, and the user's heart rate sequence can also be obtained by obtaining the continuous pulse change for relevant analysis. Therefore, the application range is quite wide and does not be limited herein. Namely, the optical sensor 12 uses photoplethysmography to measure the heartbeat interval signal. More specifically, the optical sensor is used to capture the user's heart rate physiological signal, respiratory physiological signal, blood oxygen saturation concentration physiological signal, blood pressure physiological signal and meridian blood flow distribution physiological signal. The heart rate physiological signal is the heart beat interval signal (i.e. PPG signal). The optical sensor 12 detects the heartbeat related signal of the user and transmits the detected heartbeat related signal to the microprocessor 13. In one embodiment, the heartbeat related signal detected by the optical sensor 12 is a heartbeat interval signal. In addition, other detection methods can also be used in other embodiments of the disclosure, which is not limited thereto.

In an embodiment of the disclosure, the memory module 15 can store all the information required for the operation of the apparatus 1. For example, the memory module 15 can store a reference database including an arrhythmia algorithm and available for signal processing and filtering, such as a random access memory (RAM), or an internal flash memory, or a removable memory disk to store the obtained physiological signals/information/operation results.

In an embodiment of the disclosure, the alert module 14 can receive the signal from the microprocessor 13 and perform display or feedback, and the feedback method can be auditory, visual, or tactile feedback, but is not limited thereto. In the disclosure, the alert module 14 can be a display screen, an audio device such as a speaker, a tactile warning module such as a vibration module, or a display screen, an audio device and a tactile warning module. The signals calculated, compared and determined by the microprocessor 13 as obstructive apnea can be transmitted to the alert module 14 for feedback.

As shown in FIG. 1C and in view of FIG. 2A to FIG. 2B, the apparatus 1 further comprises an upper cover 101, a lower cover 102, a charging interface 103, a display interface 104 and a control interface 105. The upper cover 101 is slidably disposed on the lower cover 102 and can cover the lower cover 102. A sensing space 106 and a sensing surface 107 are arranged between the upper cover 101 and the lower cover 102. The sensing space 106 is used for accommodating the user's finger end 20, and the motion sensor 11 and the optical sensor 12 are arranged on the sensing surface 107. When the user wears the apparatus, the apparatus 1 is fitted to the finger end 20, and the finger end 20 penetrates into the sensing space 106 to monitor obstructive sleep apnea. When in use, the user first slides the upper cover 101, extends the finger end 20 into the sensing space 106, presses down a silicone lining (not shown) in the sensing space, and covers the upper cover 101 back. The silicone lining rebounds, and then a control button of the control interface 105 is pressed to start the monitoring and evaluation of obstructive sleep apnea.

Referring to FIG. 3, a block diagram of a module of the apparatus for evaluating obstructive apnea with PPG signals according to an embodiment of the disclosure is shown. The microprocessor 13 is electrically connected to the motion sensor 11, the optical sensor 12, the alert module 14 and the memory module 15. The microprocessor 13 receives heart rate physiological signals, respiratory physiological signals, blood oxygen saturation concentration physiological signals, blood pressure physiological signals, meridian blood flow distribution physiological signals, movement detection physiological signals and/or posture sensing physiological signals, and transmits these physiological signals to an external device 17 by wireless communication through a wireless transmission module 16. The wireless transmission module 16 is wirelessly connected to at least one external device 17 to output relevant messages detected and interpreted by the device, or input information or instructions from the external device 17. Besides, wireless transmission modes of the wireless transmission module 16 include but not limited to NFC (Near Field Communication), RFID (Radio Frequency Identification), Bluetooth (Bluetooth), Infrared Communication (IrDA, Infrared Data Association), Ultra-wideband (UWB, Ultra-wideband), IEEE, Hiper LAN, and other short-range communication or medium-range communication and long-range communication methods. In addition, the external device 17 can be a smart phone, a tablet computer, a personal computer, a notebook computer, or other electronic devices or external devices, but is not limited thereto. The external device 17 receives these physiological signals and cooperates with the application program configured on it to perform operations, thereby correspondingly generating evaluation data whether the user is in a state of obstructive apnea, and the evaluation data can be displayed on the display interface 104 of the device for review.

In addition, the apparatus further includes a power management module 18, and the power management module 18 can be a battery, a power storage device, a DC power supply device, or an AC power supply device, etc., but not limited thereto, and is configured for providing the power required for the operation of the apparatus 1.

In one embodiment, the apparatus 1 further includes a heart rate sensor, which is electrically connected to the microprocessor 13. When the user is in a stationary state and is not sleeping, the heart rate sensor detects the heart rate signal of the user. The heart rate sensor is composed of a first electrode 108 and a second electrode 109. The heart rate sensor detects the heartbeat interval signal and calculates the heartbeat number. When the apparatus 1 determines that the user is in the stationary state and is not sleeping, the heart rate sensor will detect the user's heartbeat interval signal and the number of heartbeats within a predetermined time. In this embodiment, the predetermined time can be an appropriate and fixed time interval, such as 10 seconds to 5 minutes, but is not limited thereto. Then, the heart rate sensor transmits the heartbeat interval signal and heartbeat number within the predetermined time to the microprocessor 13. Next, the microprocessor 13 determines whether it is an atrial fibrillation signal. The microprocessor 13 determines whether the received signal is an atrial fibrillation signal according to the heartbeat interval signal, the number of heartbeats, the standard deviation and other information. If the microprocessor 13 determines the received signal as atrial fibrillation signal, an atrial fibrillation alert will be issued, if the microprocessor 13 determines that the received signal is not an atrial fibrillation signal, the microprocessor 13 will determine whether there is an abnormal heartbeat. In the embodiment of the disclosure, the abnormal heartbeat referred herein includes premature waves, fast heartbeats, slow heartbeats, and heartbeat signals with special rhythm. The normal heartbeat referred herein is the signal caused by the sinoatrial node. If the microprocessor 13 determines that there is no abnormal heartbeat, no alert will be issued, if the microprocessor 13 determines that there is an abnormal heartbeat, an arrhythmia alert will be issued. According to the above description, the apparatus 1 will determine in advance whether the user is in the stationary state, and when the apparatus 1 determines that the user is not in a sleep state, the apparatus 1 will start to perform a heartbeat signal detection step to detect the user's atrial fibrillation and issue an atrial fibrillation alert. For patients suffer from both atrial fibrillation (AF) and obstructive sleep apnea (OSA), even if atrial fibrillation has been cured, the probability of recurrence is much higher than that of the patients only suffering from atrial fibrillation. Therefore, in this disclosure, in different time periods and different states of the user, the synchronous monitoring of atrial fibrillation and obstructive apnea can be achieved by using optical sensors and heart rate sensors, respectively (For example, heart rate sensor is used to measure ECG signal to monitor atrial fibrillation during the day, and light sensor is used to measure heart rate interval signal (PPG signal) to monitor obstructive apnea at night.).

In one embodiment of the disclosure, the microprocessor 13 can process, calculate and interpret the signals. For example, upon receiving the heartbeat interval signal transmitted by the motion sensor 11 and the optical sensor 12, the microprocessor 13 can immediately process a signal within a predetermined period. In one embodiment, the signal processing includes a first analysis program, a second analysis program and a third analysis program.

In the first analysis program, taking 60 seconds as a signal segment, a peak to peak interval (PPI) and a PPG derived respiration (PDR) are extracted from the heartbeat interval signal (also known as PPG signal), respectively. The signal of respiratory rate analyzed from the heartbeat interval signal is the respiratory characteristic signal. The peak to peak interval (PPI) refers to the interval between peaks, which is defined as the time difference between two consecutive peaks in the heartbeat interval signal.

In the second analysis program, the cross-power spectral density of the pulse rate interval sequence and the respiration sequence is calculated to obtain a first parameter (Pxy), the coherence of the pulse rate interval sequence and the respiration sequence is calculated to obtain a second parameter (Cxy), and the spectral power density of the respiration sequence is calculated to obtain a third parameter (Py). The steps of multiplying the first parameter (Pxy) by the second parameter (Cxy) and extracting the value of the multiplication of the first parameter (Pxy) and the second parameter (Cxy) in the low frequency band and the high frequency band respectively are performed to obtain a high frequency pulse/respiration signal (HFpulse/resp) and a low frequency pulse/respiration signal (LFpulse/resp). Moreover, the third parameter (Py) in the low frequency band and the high frequency band are extracted respectively to obtain a high frequency respiration signal (HFresp) and a low frequency respiration signal (LFresp).

In the third analysis program, the signals in different frequency bands (such as the low frequency band and high frequency band) are performed a logarithmic process. Namely, a ratio of the low frequency pulse/respiration signal to the high frequency pulse/respiration signal (LFpulse/resp/HFpulse/resp) is multiplied by a ratio of the low frequency respiration signal to the high frequency respiration signal (LFresp/HFresp) and performed the logarithmic process. The logarithmic processing is to take the natural logarithm (In) of a ratio of the multiplied low frequency pulse/respiration signal to the high frequency pulse/respiration signal (LFpulse/resp/HFpulse/resp) and a ratio of the low frequency respiration signal to the high frequency respiration signal (LFresp/HFresp) to obtain the apnea parameter. The apnea parameter is compared with a threshold in a normal sleep respiration database to determine whether the heartbeat interval signal is an obstructive apnea signal, and further to determine whether the user is in an obstructive apnea condition.

In one embodiment, a range of the threshold is between −3 and −5. If the obtained apnea parameter is greater than the range specified by the threshold, it is determined that the user is in the state of obstructive apnea, and the alert module of the apparatus can issue an alert to inform the user.

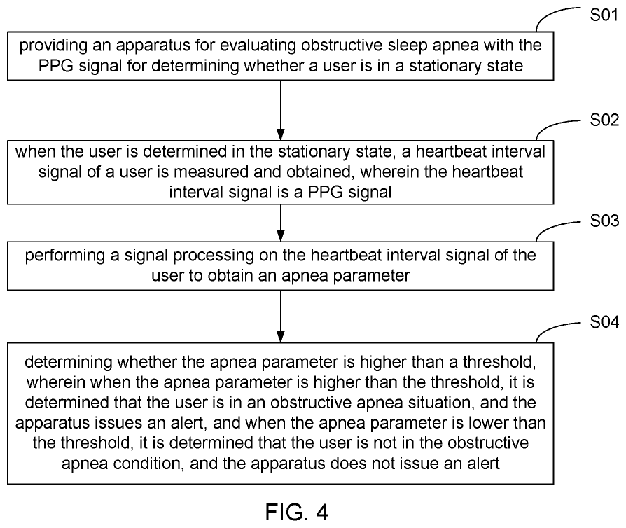

Referring to FIG. 4, a schematic flowchart of a method for evaluating obstructive apnea with PPG signals according to the disclosure is shown. The method for evaluating obstructive sleep apnea with a PPG signal comprises the following steps.

In a step S01, an apparatus for evaluating obstructive sleep apnea with the PPG signal is provided for determining whether a user is in a stationary state.

In a step S02, when the user is determined in the stationary state, a heartbeat interval signal of a user is measured and obtained, and the heartbeat interval signal is a PPG signal.

In a step S03, a signal processing is performed on the heartbeat interval signal of the user to obtain an apnea parameter.

The signal processing comprises: a first analysis program, a second analysis program and a third analysis program.

In the first analysis program, taking 60 seconds as a signal segment, a peak to peak interval (PPI) and a PPG derived respiration (PDR) are extracted from the heartbeat interval signal (also known as PPG signal), respectively. The signal of respiratory rate analyzed from the heartbeat interval signal is the respiratory characteristic signal. The peak to peak interval (PPI) refers to the interval between peaks, which is defined as the time difference between two consecutive peaks in the heartbeat interval signal.

In the second analysis program, the cross-power spectral density of the pulse rate interval sequence and the respiration sequence is calculated to obtain a first parameter (Pxy), the coherence of the pulse rate interval sequence and the respiration sequence is calculated to obtain a second parameter (Cxy), and the spectral power density of the respiration sequence is calculated to obtain a third parameter (Py). The steps of multiplying the first parameter (Pxy) by the second parameter (Cxy) and extracting the value of the multiplication of the first parameter (Pxy) and the second parameter (Cxy) in the low frequency band and the high frequency band respectively are performed to obtain a high frequency pulse/respiration signal (HFpulse/resp) and a low frequency pulse/respiration signal (LFpulse/resp). Moreover, the third parameter (Py) in the low frequency band and the high frequency band are extracted respectively to obtain a high frequency respiration signal (HFresp) and a low frequency respiration signal (LFresp).

In the third analysis program, the signals in different frequency bands are performed a logarithmic process. That is to say, a ratio of the low frequency pulse/respiration signal to the high frequency pulse/respiration signal (LFpulse/resp/HFpulse/resp) is multiplied by a ratio of the low frequency respiration signal to the high frequency respiration signal (LFresp/HFresp) and performed the logarithmic process. The logarithmic processing is to take the natural logarithm (In) of a ratio of the multiplied low frequency pulse/respiration signal to the high frequency pulse/respiration signal (LFpulse/resp/HFpulse/resp) and a ratio of the low frequency respiration signal to the high frequency respiration signal (LFresp/HFresp) to obtain the apnea parameter. The apnea parameter is compared with a threshold in a normal sleep respiration database to determine whether the heartbeat interval signal is an obstructive apnea signal, and further to determine whether the user is in an obstructive apnea condition.

a third analysis program for multiplying a ratio of the low frequency pulse/respiration signal to the high frequency pulse/respiration signal (LFpulse/resp/HFpulse/resp) and a ratio of the low frequency respiration signal to the high frequency respiration signal (LFresp/HFresp) and performing a logarithmic processing to obtain the apnea parameter, and for comparing the apnea parameter with a threshold in a normal sleep respiration database to determine whether the heartbeat interval signal is an obstructive apnea signal, and further determine whether the user is in an obstructive apnea condition; and

In a step S04, whether the apnea parameter is higher than a threshold is determined. When the apnea parameter is higher than the threshold, it is determined that the user is in an obstructive apnea situation, and the apparatus issues an alert, and when the apnea parameter is lower than the threshold, it is determined that the user is not in the obstructive apnea condition, and the apparatus does not issue the alert.

In one embodiment, a range of the threshold is between −3 and −5. If the obtained apnea parameter is greater than the range specified by the threshold, it is determined that the user is in the state of obstructive apnea, and the alert module of the apparatus can issue an alert to inform the user.

In one embodiment, the high frequency pulse/respiration signal and the high frequency respiration signal are signals with a frequency greater than or equal to 0.15 Hz and less than 0.4 Hz, and the low frequency pulse/respiration signal and the low frequency respiration signal are signals with a frequency greater than or equal to 0.04 Hz and less than 0.15 Hz.

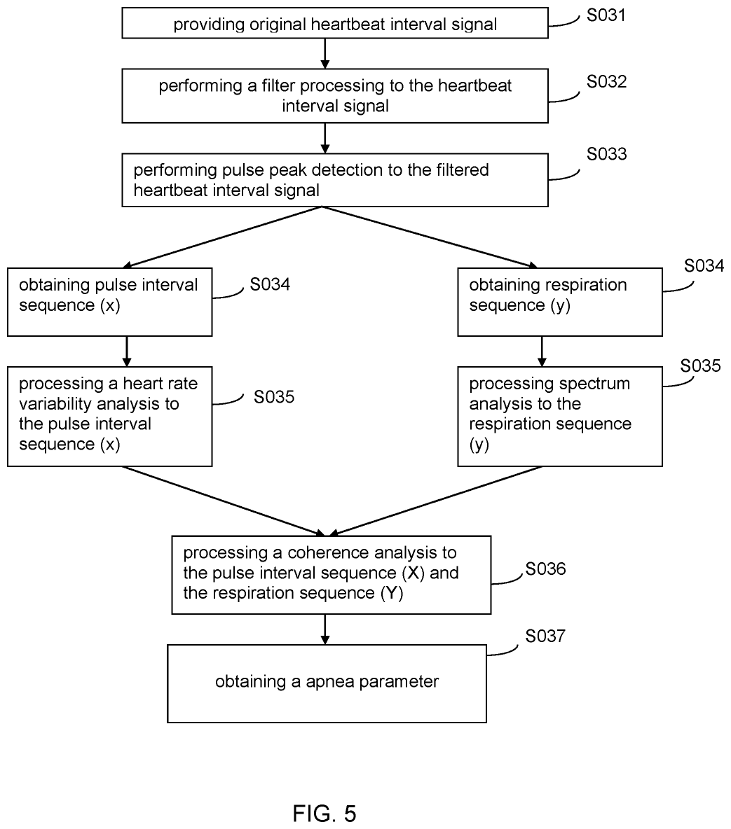

Referring to FIG. 5, a schematic flowchart of a signal processing step of the method for evaluating obstructive apnea with PPG signals according to the disclosure is shown. In the step of the signal processing as the above-mentioned step S03, the following sub-steps are further included.

In a step S031, original heartbeat interval signals are provided. For example, the microprocessor receives the heartbeat interval signal captured by the optical sensor, which is the PPG signal.

In a step S032, the heartbeat interval signal is performed a filter processing.

In a step S033, the filtered heartbeat interval signal is subjected to pulse peak detection (PD).

In a step S034, pulse rate interval sequence (x) and respiration sequence (y) are obtained.

In a step S035, heart rate variability analysis was performed on the pulse rate interval sequence (x) and spectral analysis was performed on the respiration sequence (y).

In a step S036, a coherence analysis is processed to the pulse interval sequence (x) and the respiration sequence (y). The coherence analysis is the measurement of the connection value or coupling value between two different time periods. Therefore, the coherence of the pulse rate interval sequence and the respiration sequence in the low frequency band and in the high frequency band can be calculated, respectively. Accordingly, the correlation between the pulse rate interval sequence and the respiration sequence can be calculated in the low frequency band and in the high frequency band, respectively.

In a step S037, the apnea parameter is obtained.

As mentioned above, the apnea parameter can be obtained by photoplethysmography (PPG) through two different analysis methods: time domain analysis and frequency domain analysis. Time domain analysis is a statistical analysis of continuous pulse interval (pulse rate interval). The variation of pulse rate is presented by statistical index. The frequency domain analysis is then performed to obtain the total power that can be used to evaluate the overall heart rate variability. High frequency power (HF) signal that can reflect the activity of parasympathetic nerve, low frequency power (LF) signal that can reflect the activity of sympathetic nerve or the result of simultaneous regulation of sympathetic nerve and parasympathetic nerve, and LF/HF (low/high frequency power ratio) that can reflect the activity balance of sympathetic/parasympathetic activity. Therefore, the filter processing is performed first, and pulse peak detection (PD) is used to obtain the pulse rate interval sequence and the respiration sequence. The cross-power spectral density (Pxy) and the coherence (Cxy) of the pulse rate interval sequence (x) and the respiration sequence (y) are calculated. The obtained parameters Pxy and Cxy are multiplied, and the multiplied values of Pxy and Cxy are extracted in the low frequency band and high frequency band, respectively, to obtain the high frequency pulse/respiration signal (HFpulse/resp) and the low frequency pulse/respiration signal (LFpulse/resp). Similarly, the spectral power (Py) of the respiration sequence (y) is calculated. The value of the spectral power (Py) of the respiration sequence (y) in the low frequency band and the high frequency band, respectively, are extracted to obtain a high frequency respiration signal (HFresp) and a low frequency respiration signal (LFresp). The apnea parameter is obtained by multiplying the ratio of LFpulse/resp/HFpulse/resp by the ratio of LFresp/HFresp and taking the natural logarithm.

It should be noted that the frequency band of heart rate variability is divided into extremely low frequency (VLF, 0˜0.04 Hz) signal, low frequency (LF, 0.04˜0.15 Hz) signal and high frequency (HF, 0.15˜0.40 Hz) signal. Extremely low frequency signals are associated with heat regulation and fluid regulation. Low frequency signals reflect the co-regulation of sympathetic and parasympathetic nerves on heart rate and the increase in power is usually considered to be the result of sympathetic nerve activity. High frequency signal is related to respiration, mainly reflecting the regulation of the heart rate by the parasympathetic nerves. The ratio of the low frequency pulse/ respiration signal to the high frequency pulse/respiration signal obtained in the low frequency band and the high frequency band, respectively, mainly reflects the state of the sympathetic nervous system and the parasympathetic nervous system and the changing trend of their balance. The cross-power spectral density analysis of pulse rate interval sequence is mainly to study the activity of sympathetic and parasympathetic nerves in different sleep phases.

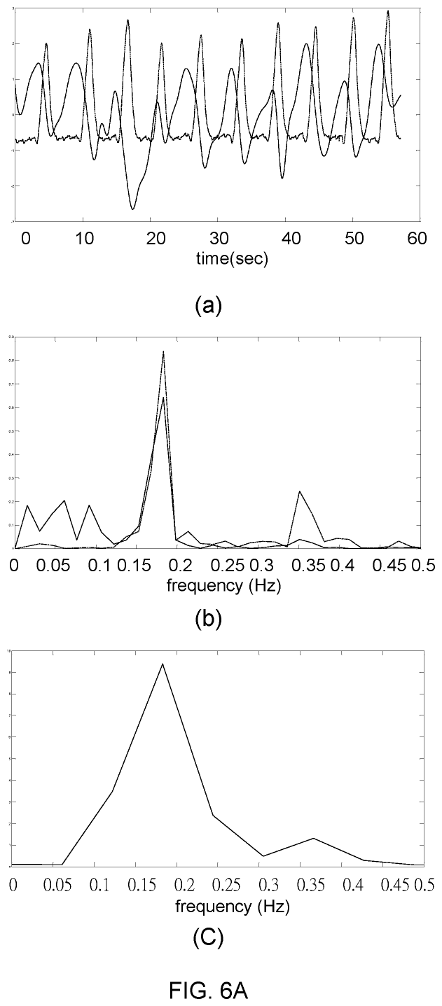

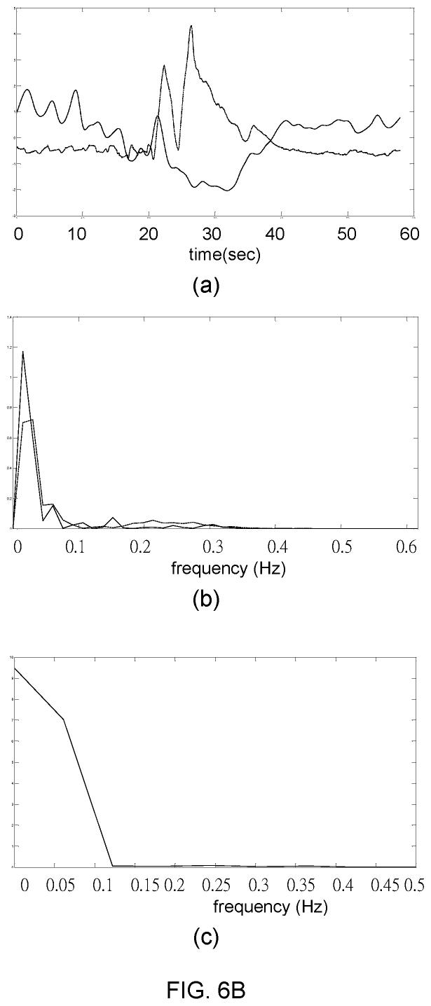

Referring to FIG. 6A and FIG. 6B, the apparatus for evaluating obstructive apnea with PPG signals to measure physiological signals of a user under a normal sleep respiration condition (FIG. 6A) and a obstructive apnea condition (FIG. 6B), respectively, parts (a) in FIG. 6A and FIG. 6B represent pulse rate interval sequence (solid line) and respiration sequence (broken line), parts (b) represent pulse rate interval sequence spectrum (solid line) and respiration sequence spectrum (broken line), and parts (c) represent the product of the cross-power spectral density of the pulse rate interval sequence and the respiration sequence and the coherence of the pulse rate interval sequence and the respiration sequence are shown. In FIG. 6A, after the microprocessor operation, the ratio of low frequency pulse/respiration signal (LFpulse/resp) and high frequency pulse/respiration signal (HFpulse/resp) is taken as the natural logarithm and the value is −0.12. The ratio of the low frequency respiration signal (LFresp) to the high frequency respiration signal (HFresp) is −3.43 after taking the natural logarithm. After multiplying the ratio of LFpulse/resp and HFpulse/resp and the ratio of LFresp and HFresp, the obtaoned value after taking the natural logarithm is −3.56, which is not higher than the range between −3 and −5 defined by the threshold. Therefore, when the apnea parameter is lower than the threshold, the apparatus determines that the user is in normal sleep and breathing, and the apparatus does not issue an alert. In FIG. 6B, the ratio of low frequency pulse/ respiration signal (LFpulse/resp) and high frequency pulse/respiration signal (HFpulse/resp) is −3.49 after taking the natural logarithm. The ratio of the low frequency respiration signal (LFresp) to the high frequency respiration signal (HFresp) is −3.51 after taking the natural logarithm. The ratio of LFpulse/resp and HFpulse/resp and the ratio of LFresp and HFresp are multiplied and taken the natural logarithm to obtain a value, the value is −7.00. It can be seen that the apnea parameter is above the range of the threshold. When the apparatus determines that the apnea parameter is higher than the threshold, and determines that the user is in an obstructive apnea situation, the apparatus issues an alert.

The disclosure provides an apparatus and method for evaluating obstructive apnea with PPG signal. For users suffer from both atrial fibrillation and obstructive apnea, the atrial fibrillation and the obstructive apnea can be detected and evaluated simultaneously after the atrial fibrillation is cured. For users suffering from both atrial fibrillation and obstructive apnea, after the atrial fibrillation has been cured, the probability of recurrence is much higher than that of patients with obstructive apnea only. Therefore, the disclosure provides the apparatus and method for evaluating obstructive apnea with PPG signal for simultaneously monitoring of atrial fibrillation and obstructive apnea with optical sensor and heart rate sensor, respectively (For example, when the user is in the stationary state and is not sleeping during the day, the heart rate sensor is used to measure the ECG signal to monitor atrial fibrillation, and when the user is in the stationary state and is sleeping at night, the optical sensor is used to measure the heartbeat interval signal (PPG signal) to monitor the obstructive apnea).

The description of the above embodiments is only used to understand the method and features of this disclosure. The present disclosure has been described with preferred embodiments thereof and it is understood that many changes and modifications to the described embodiment can be carried out without departing from the scope and the spirit of the invention that is intended to be limited only by the appended claims.

What is claimed is:

1. An apparatus for evaluating obstructive sleep apnea with a PPG signal, comprising:
a motion sensor configured to detect whether a user is in a stationary state;
an optical sensor configured to measure a heartbeat interval signal of the user in the stationary state, wherein the heartbeat interval signal is the PPG signal;
a microprocessor, electrically connected to the motion sensor and the optical sensor, respectively, and configured to perform a signal processing on the heartbeat interval signal of the user to obtain an apnea parameter;
an alert module, electrically connected to the microprocessor, configured to receive the apnea parameter and configured to issue an alert; and
a memory module electrically connected to the microprocessor and configured to store the apnea parameter after the signal processing;
wherein the signal processing comprises:
a first analysis program for extracting a pulse rate interval sequence and a respiration sequence from the heartbeat interval signal, respectively;
a second analysis program for calculating a cross-power spectral density of the pulse rate interval sequence and the respiration sequence to obtain a first parameter, calculating a coherence between the pulse rate interval sequence and the respiration sequence to obtain a second parameter, and calculating a spectral power of the respiration sequence to obtain a third parameter, wherein the first parameter and the second parameter are multiplied, and a multiplied value of the first parameter and the second parameter in a low frequency band and a high frequency band, respectively, are extracted to obtain a high frequency pulse/respiration signal (HFpulse/resp) and a low frequency pulse/respiration signal (LFpulse/resp), and values of the third parameter in the low frequency band and the high frequency band, respectively, are extracted to obtain a high frequency respiration signal (HFresp) and a low frequency respiration signal (LFresp); and
a third analysis program for multiplying a ratio of the low frequency pulse/respiration signal to the high frequency pulse/respiration signal (LFpulse/resp/HFpulse/resp) and a ratio of the low frequency respiration signal to the high frequency respiration signal (LFresp/HFresp) and performing a logarithmic processing to obtain the apnea parameter, and for comparing the apnea parameter with a threshold in a normal sleep respiration database to determine whether the heartbeat interval signal is an obstructive apnea signal, and further determine whether the user is in an obstructive apnea condition.

2. The device according to claim 1, wherein the device further comprises a heart rate sensor electrically connected to the microprocessor, when the user is in a non sleep static state, the heart rate sensor detects the heart rate signal of the user.

3. The device according to claim 1, wherein the high frequency pulse/respiration signal and the high frequency respiration signal are signals with a frequency greater than or equal to 0.15 Hz and less than 0.4 Hz, and the low frequency pulse/respiration signal and the low frequency respiration signal are signals with a frequency greater than or equal to 0.04 Hz and less than 0.15 Hz.

4. The device according to claim 1, wherein the logarithmic processing comprises multiplying the ratio of the low frequency pulse/respiration signal to the high frequency pulse/respiration signal (LFpulse/resp/HFpulse/resp) and the ratio of the respiratory low frequency respiration signal to the high frequency respiration signal (LFresp/HFresp) and taking a natural logarithm to obtain the apnea parameter.

5. The device according to claim 1, wherein a value range of the threshold is between −3 and −5.

6. A method for evaluating obstructive sleep apnea with a PPG signal, comprising steps of:
a step S01 of providing an apparatus for evaluating obstructive sleep apnea with the PPG signal for determining whether a user is in a stationary state;
a step S02 that when the user is determined in the stationary state, a heartbeat interval signal of a user is measured and obtained, wherein the heartbeat interval signal is a PPG signal;
a step S03 of performing a signal processing on the heartbeat interval signal of the user to obtain an apnea parameter;
wherein the signal processing comprises:
a first analysis program for extracting a pulse rate interval sequence and a respiration sequence from the heartbeat interval signal, respectively;
a second analysis program for calculating a cross-power spectral density of the pulse rate interval sequence and the respiration sequence to obtain a first parameter, calculating a coherence between the pulse rate interval sequence and the respiration sequence to obtain a second parameter, and calculating a spectral power of the respiration sequence to obtain a third parameter, wherein the first parameter and the second parameter are multiplied, and a multiplied value of the first parameter and the second parameter in a low frequency band and a high frequency band, respectively, are extracted to obtain a high frequency pulse/respiration signal (HFpulse/resp) and a low frequency pulse/respiration signal (LFpulse/resp), and values of the third parameter in the low frequency band and the high frequency band, respectively, are extracted to obtain a high frequency respiration signal (HFresp) and a low frequency respiration signal (LFresp); and
a third analysis program for multiplying a ratio of the low frequency pulse/respiration signal to the high frequency pulse/respiration signal (LFpulse/resp/HFpulse/resp) and a ratio of the low frequency respiration signal to the high frequency respiration signal (LFresp/HFresp) and performing a logarithmic processing to obtain the apnea parameter, and for comparing the apnea parameter with a threshold in a normal sleep respiration database to determine whether the heartbeat interval signal is an obstructive apnea signal, and further determine whether the user is in an obstructive apnea condition; and
a step S04 of determining whether the apnea parameter is higher than a threshold, wherein when the apnea parameter is higher than the threshold, it is determined that the user is in an obstructive apnea situation, and the apparatus issues an alert, and when the apnea parameter is lower than the threshold, it is determined that the user is not in the obstructive apnea condition, and the apparatus does not issue an alert.

7. The method according to claim 6, wherein the device further comprises a heart rate sensor electrically connected to the microprocessor, when the user is in a non sleep static state, the heart rate sensor detects the heart rate signal of the user.

8. The method according to claim 6, wherein the high frequency pulse/respiration signal and the high frequency respiration signal are signals with a frequency greater than or equal to 0.15 Hz and less than 0.4 Hz, and the low frequency pulse/respiration signal and the low frequency respiration signal are signals with a frequency greater than or equal to 0.04 Hz and less than 0.15 Hz.

9. The method according to claim 6, wherein the logarithmic processing comprises multiplying the ratio of the low frequency pulse/respiration signal to the high frequency pulse/respiration signal (LFpulse/resp/HFpulse/resp) and the ratio of the respiratory low frequency respiration signal to the high frequency respiration signal (LFresp/HFresp) and taking a natural logarithm to obtain the apnea parameter.

10. The method according to claim 6, wherein a value range of the threshold is between −3 and −5.
